(19) United States
(12) Patent Application Publication
(10) Pub. No.: US 2021/0217166 A1
(43) Pub. Date: Jul. 15, 2021

(54) AUTOMATED SCREENING OF MEDICAL DATA

(71) Applicant: Visionairy Health, Inc., Fort Lee, NJ (US)

(72) Inventors: Moritz Alexander Graule, Cambridge, MA (US); Moustafa Mohamed Amin, New Haven, CT (US); Matthias Konrad Bloch, Schaffhausen (CH)

(73) Assignee: Visionairy Health, Inc., Fort Lee, NJ (US)

(21) Appl. No.: 17/054,747

(22) PCT Filed: May 10, 2019
(86) PCT No.: PCT/US2019/031839

§ 371 Date: Nov. 11, 2020

Related U.S. Application Data
(60) Provisional application No. 62/670,119, filed on May 11, 2018.

(51) Int. Cl. G06T 7/00; G06K 9/00; G06K 9/46; G16H 10/60; G16H 30/20; G16H 30/40; G16H 50/70; G16H 40/20

(52) U.S. Cl. CPC G06T 7/0014; G06K 9/00369; G06K 9/4604; G16H 10/60; G16H 30/20; G16H 30/40; G16H 50/20; G16H 40/20; G06K 2209/01; G06K 2209/055; G06T 2207/30168; G06T 2207/30008; G16H 50/70

(57) ABSTRACT

Methods are provided for expediting screening of medical data. In some methods, medical records are obtained from a requester. Each medical record includes a digitized image of a body region of a patient. For each image, a processor is used to: perform an image-quality check of the image; perform a character-recognition process to locate a character in the image; mask the character to obtain a masked image; perform an identification process on the masked image to identify the body region of the image; perform an analysis routine on the masked image to determine a screening score, the analysis routine corresponding to the identified body region; and, if the screening score is within a normal range for the analysis routine, generate a normal notification indicating that the image is a normal image within a healthy range for the identified body region. The normal notification is automatically transmitted to a requester.

### CROSS REFERENCE TO RELATED APPLICATION

The present application claims the benefit of priority of U.S. Provisional Application No. 62/670,119, filed in the U.S. Patent and Trademark Office on May 11, 2018, the entire disclosure of which is incorporated by reference herein.

### FIELD OF THE DISCLOSURE

The technology disclosed herein relates generally to electronic processing of medical data. More particularly, the present technology relates to using algorithms to identify information from medical data and to use the information to automate and expedite screening of medical data.

### BACKGROUND

Health care costs have increased significantly in recent decades. Some of the increased costs may be due to the increased use of testing to diagnose ailments. The testing may require not only the use of sophisticated and complex machinery to obtain test data, but also may require experts or trained personnel to analyze the data and return test results or reports in which an evaluation of the data is provided. Moreover, patients and physicians often are eager to obtain such reports quickly, thus necessitating the trained personnel to work long hours or a greater number of trained personnel to be staffed, in order to perform the analyses and prepare the reports in a timely manner.

FIG. 1 shows a flow chart summarizing a conventional clinical workflow for medical testing of patients. Initially, at S10, a request is made for an imaging study of a patient, so that, for example, images of a body region of the patient may be studied more closely. The request may be made by, for example, a physician (e.g., a pulmonologist). The requester may send the request to an image acquisition facility (e.g., a radiology laboratory) for an imaging study to be performed on the patient. The requester also may send additional information to the radiology laboratory. For example, the additional information sent by the requester may be information about the patient and/or instructions regarding the desired region(s) of the patient to be studied. At S15, the radiology laboratory also may receive additional information from a third-party source other than the requester. For example, the requester may instruct the patient's primary care physician (“PCP”) and/or the patient's health insurance provider to provide information directly to the radiology laboratory.

At S20, the radiology laboratory performs the imaging study on the patient. The imaging study may include at least one image of the patient.

The imaging study and the additional information may be provided to trained personnel, such as a radiologist or another expert having expertise in, for example, lungs. At S30, the imaging study is analyzed by the trained personnel. The additional information from the requester and/or the third party may be used by the trained personnel to perform the analysis.

At S40, analysis results are compiled into a report by the trained personnel, and, at S50, the report is provided to the requester. The report may include findings or observations about the imaging study (e.g., a spot on a mammogram; a shadow on an x-ray image of a lung; an absence of an abnormality; etc.). The report also may include a diagnosis (e.g., cancer; pneumonia; healthy specimen; etc.).

### SUMMARY OF THE DISCLOSURE

Aspects of the present technology are directed to streamlining procedures involved in medical testing. To this end, systems and methods are provided that may be used to process electronic data obtained from imaging studies as well as other diagnostic studies and evaluative medical procedures.

Some aspects of the present technology may utilize machine-learning techniques and/or algorithms to streamline and expedite one or more of the procedures involved in medical testing, such as identification and/or extraction of specific features from medical data, and correlation of those features to medical observations and even diagnoses.

Some aspects of the present technology may expedite evaluative processing of an imaging study in which, with a high degree of certainty, no detectable abnormality was found. For such a healthy imaging study, a notification (“normal notification”) may be automatically generated indicating that no abnormal or unusual feature was found for the body region of the study, and the notification may be automatically sent to a facility or an individual physician (collectively referred to as “requester” herein) who requested evaluative processing of the imaging study. The healthy imaging study may be automatically eliminated from the clinical workflow queue and thus may bypass human evaluation by trained personnel (e.g., a radiologist, a specialist in the body region of the study, etc.). Expedited elimination of healthy imaging studies from the workflow queue may consequently enable the trained personnel to have more time to evaluate other imaging studies in the workflow queue. In an aspect, the remaining imaging studies, which were not automatically eliminated from the workflow queue, may undergo a second tier of streamlining, in which a non-specialist clinician (e.g., a trained nurse) performs an evaluation of the image. Only if the non-specialist clinician deems the image to show an abnormality or an unusual feature does the image undergo evaluation by a specialist (e.g., a radiologist). In this aspect, if the non-specialist clinician deems the image to show a normal, healthy body region, the image may be removed from the workflow queue and a normal notification may be provided to the requester.

According to an aspect of the present technology, a method for expediting screening of medical data is provided. In the method, medical records from a requester are electronically obtained. Each of the medical records may comprise a digitized image of a body region of a patient. For each of the images, a computer processor is used to: perform a character-recognition process to locate at least one character in the image, the at least one character being one of or a combination of: a symbol character and a text character; masking each of the located at least one character, to obtain a masked image; performing an identification process on the masked image to identify the body region of the image; performing an analysis routine on the masked image to determine a screening score, the analysis routine corresponding to the identified body region; and, if the screening score determined by the analysis routine is within a normal range for the analysis routine, generate a normal notification indicating that the image is a normal image within a healthy range for the identified body region. The normal notification is automatically transmitted to the requester. A system and a non-transitory computer-readable storage medium also are provided.

According to another aspect of the present technology, a computer-implemented method for screening medical data is provided. In the method, medical records provided by a requester are obtained. Each of the medical records includes an image of a body region of a patient. For each of the images, a computer processor is utilized to: perform automatically an identification process on the image to identify the body region; select automatically an analysis routine to analyze the identified body region; analyze automatically the medical image using the selected analysis routine to calculate a screening score corresponding to the image; and, if the screening score corresponding to the image is equal to or below a predetermined threshold for the selected analysis routine, generate automatically a normal notification indicating that the image is a normal image within a healthy range for the identified body region. A system and a non-transitory computer-readable storage medium also are provided.

### BRIEF DESCRIPTION OF DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 shows a flowchart for a conventional workflow.

FIGS. 2A-2D show flowcharts for a workflow for evaluating an image, according to an aspect of the present technology.

FIGS. 3A-3D show an image of a pair of lungs at various stages of a screening process, according to an aspect of the present technology.

FIGS. 4A and 4B show an image of a pair of lungs and a heart at various stages of a screening process, according to an aspect of the present technology.

FIG. 5A shows an x-ray image of a male patient.

FIG. 5B shows an x-ray image of a female patient.

FIGS. 6A and 6B show x-ray images with characters and/or graphics.

FIG. 7 shows a flowchart for a workflow for processing characters and/or graphics in an image, according to an aspect of the present technology.

FIGS. 8A-8C show an image of a pair of lungs at various stages of processing for characters and/or graphics, according to an aspect of the present technology.

FIGS. 9A-9C show another image of a pair of lungs at various stages of processing for characters and/or graphics, according to an aspect of the present technology.

FIG. 10 schematically shows a computer system, according to an aspect of the present technology.

FIG. 11 schematically shows an operating environment of the computer system of FIG. 10, according to an aspect of the present technology.

FIG. 12 shows examples of different types of textures, in another aspect of the present technology.

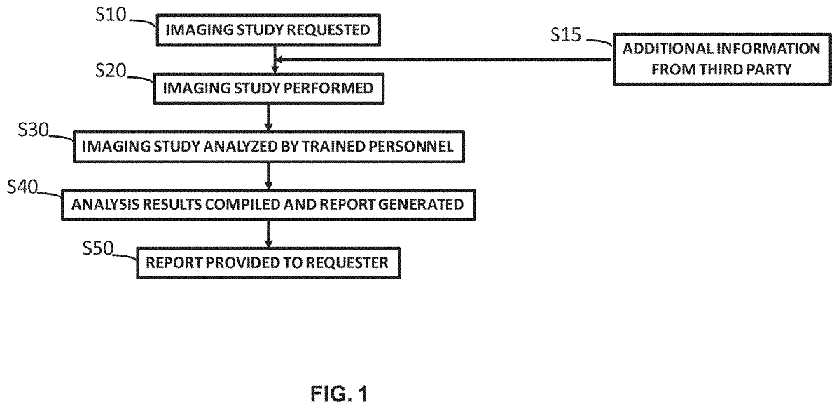

### DETAILED DESCRIPTION

Methods are provided for expediting screening of medical data. In some methods, medical records are obtained from a requester. Each medical record includes a digitized image of a body region of a patient. For each image, a processor is used to: perform an image-quality check of the image; perform a character-recognition process to locate a character in the image; mask the character to obtain a masked image; perform an identification process on the masked image to identify the body region of the image; perform an analysis routine on the masked image to determine a screening score, the analysis routine corresponding to the identified body region; and, if the screening score is within a normal range for the analysis routine, generate a normal notification indicating that the image is a normal image within a healthy range for the identified body region. The normal notification is automatically transmitted to a requester.

Some aspects of the present technology are directed to streamlining procedures involved in medical testing. To this end, systems and methods are provided that may be used to process electronic data obtained from imaging studies as well as other diagnostic studies and evaluative medical procedures.

Some aspects of the present technology may utilize machine-learning techniques and/or algorithms to identify and/or extract specific features from medical data, and to correlate those features to medical observations and even diagnoses.

Some aspects of the present technology may expedite evaluative processing of an imaging study in which, with a high degree of certainty, no detectable abnormality was found. For such a healthy imaging study, a notification may be generated automatically and may indicate that no abnormal or unusual feature was found for the body region of the imaging study. The notification may be sent automatically to a facility or an individual who requested the imaging study. The healthy imaging study may be automatically eliminated from the workflow queue and thus may bypass human evaluation by trained personnel, thus reducing the number of cases to be handled by the trained personnel. In an aspect, the remaining imaging studies, which were not automatically eliminated from the workflow queue, may undergo a second tier of streamlining, in which a non-specialist clinician performs an evaluation of the image. Only if the non-specialist clinician confirms that the image shows an abnormality or an unusual feature does the image undergo evaluation by a specialist (e.g., a radiologist). In this aspect, if the non-specialist clinician deems the image to show a normal, healthy body region, the image may be removed from the workflow queue and a normal notification may be provided to the requester.

The normal notification may be a full report (e.g., a text document) summarizing the evaluation(s) performed; or may be a coded symbol (e.g., by color or shape) or a flag appended to the image or its corresponding medical report; or may be an absence of a flag or coded symbol on the image or its corresponding medical report. As will be appreciated, a notification may take any form, as long as personnel receiving the notification is aware of how to interpret the notification.

According to an aspect of the present technology, a stream of electronic medical data transmitted by a requester may be received by a computer system of a medical evaluation facility. The medical data may be transmitted via a communication network (e.g., the Internet, a private network, etc.). The stream may include, for example, one or a plurality of digital images corresponding to one or a plurality of imaging studies submitted by the requester for medical evaluation or analysis. For example, the requester may request the medical evaluation facility to evaluate the images of the imaging studies to determine whether, for each of the images, the image shows any feature that would indicate a possible medical issue, and, if so, to identify the possible medical issue.

The system may include at least one computer processor coupled to at least one memory. The processor(s) may be specially programmed to execute one or more algorithms to process each image of the stream to determine an image-quality factor and, if the image-quality factor is above a threshold value indicating that the image is of sufficiently high quality for a reliable medical evaluation, to identify one or more abnormalities, if any, in the image.

Each imaging study may include one or more images. An imaging study in which each image of the study is determined, with a high degree of certainty, to be of sufficiently high quality and devoid of an abnormality or an unusual feature, i.e., a healthy imaging study, may be diverted from for workflow queue for expedited processing. A normal notification may be automatically generated for each healthy imaging study, and the notification may be transmitted automatically to the requester individually or collectively with other normal notifications resulting from evaluations requested by the requester.

Optionally, instead of receiving the medical data streamed directly from the requester, the medical data may be obtained by the system by accessing a memory in which the medical data is stored by the requester. For example, the requester may upload the medical data to a memory that is accessible by the system, and the system may retrieve the medical data periodically (e.g., every 24 hours, every hour, every minute, every few seconds, etc.) or when the system receives a ping message indicating that new medical data has been uploaded.

### EMBODIMENT 1

According to a first embodiment of the present technology, a method for expediting screening of medical data is provided. In the method, medical records from a requester may be obtained electronically. Each of the medical records may comprise a digitized image of a body region of a patient. For each of the images, a computer processor may be used to: perform a character-recognition process to locate at least one character in the image; mask each of the located at least one character, to obtain a masked image; perform an identification process on the masked image to identify the body region of the image; perform an analysis routine on the masked image to determine a screening score; and, if the screening score determined by the analysis routine is within a normal range for the analysis routine, generate a normal notification indicating that the image is a normal image within a healthy range for the identified body region. The normal notification may be automatically transmitted to the requester. The at least one character may be one of or a combination of: a symbol character and a text character. The analysis routine may correspond to the identified body region.

According to an aspect of the embodiment, the medical records may be obtained by receiving an electronic transmission from the requester.

According to another aspect of the embodiment, the medical records may be obtained by retrieving the medical records from a memory. The requester may store the medical records in the memory, to enable the medical records to be retrieved.

According to an aspect of the embodiment, the analysis routine may determine the screening score by combining a plurality of sub-scores resulting from a plurality of sub-routines of the analysis routine.

According to an aspect of the embodiment, the normal notification may be transmitted to the requester together with other normal notifications generated from the received medical records.

According to an aspect of the embodiment, the performing of the character-recognition process to locate the at least one character in the image may recognize the at least one character in the image. For example, the character-recognition process may recognize the at least one character in the image to be alphanumeric text corresponding to the image, and the alphanumeric text may, e.g., be a word or a string of words corresponding to the image.

According to some aspects of the embodiment, the analysis routine may comprise: identifying indicator information, obtaining first and second information from the indicator information, processing the image using the first information to determine a first factor, processing the image using the second information to determine a second factor, and calculating the screening score using at least the first and second factors.

In an aspect of the embodiment, the first information of the indicator information may comprise image-quality information, and the second information of the indicator information may comprise the body region identified in the identification process. The at least one character recognized in the character-recognition process may be utilized in the identification process to identify the body region of the image. Alternatively, the at least one character recognized in the character-recognition process may not be utilized in the identification process to identify the body region of the image.

In an aspect of the embodiment, the identification process may perform an object-contour routine on the image to identify contours of at least one object in the image. To identify the body region, the identification process may perform a comparison routine to compare the contours or a portion of the contours of the at least one object in the image with one or more reference images stored in a memory accessible by the computer processor.

In an aspect of the embodiment, the first factor may be determined by comparing the first information to first reference information stored in a database accessible by the computer processor, and the second factor may be determined by comparing the second information to second reference information stored in a database accessible by the computer processor.

According to an aspect of the embodiment, the method may further comprise evaluating the image to determine the image-quality information. The image-quality information may comprise a quality value for any one of or any combination of: a level of blurriness, evidence of patient movement, an appropriateness of magnification, a correctness of an imaging view, a presence of a non-patient artifact, an image-digitization artifact, and an improper exposure condition.

According to an aspect of the embodiment, each of the medical records may further comprise additional information. The additional information may comprise any one of or any combination of: a related previous medical image of the patient, a medical history of the patient, demographic information of the patient, a previous diagnosis of the patient, a physician comment regarding the patient, and an upcoming medical test of the patient. The analysis routine may take into account the additional information to determine the screening score.

According to an aspect of the embodiment, the second information may comprise a type of the image. The type of the image may comprise one of: an x-ray radiographic image, an ultrasound sonographic image, a magnetic-resonance imaging image, an endoscopic photograph, an epidermal photograph, and a nuclear emission radiographic image.

According to another aspect of the embodiment, the second information may comprise a category of the image. The category may be any one of or any combination of: a two-dimensional image, a three-dimensional image, a surface image, a cross-sectional image, and a tomographic image in a set of tomographic images.

According to an aspect of the embodiment, the second information may be obtained from the image or from additional information obtained by the computer processor separately from the medical records.

According to an aspect of the embodiment, the processing using the first information may determine an image-quality score for the first factor, and may compare the image-quality score to a threshold value above which a reliable analysis cannot be made from the image. The method may further comprise generating and transmitting to the requester a rejection notification indicating that the image corresponding to the image-quality score was rejected from analysis due to low image quality, when the image-quality score is above the threshold value.

According to an aspect of the embodiment, the processing using the second information may commence after the image-quality score is determined to be at or below the threshold value.

According to an aspect of the embodiment, the processing using the second information may comprise determining the image to be any one or any combination of: an external body region, an internal body region, an external body part, an internal body part, an internal organ, an implanted object (e.g., pacemaker, artificial hip, etc.), a prosthetic device, and a skeletal part.

According to an aspect of the embodiment, the processing using the second information may comprise determining whether the image includes an anomaly. The anomaly may comprise any one of or any combination of: a bone fracture, a joint dislocation, an abnormal surface contour of an internal organ, an abnormal surface texture of an external body part, an abnormal surface texture of an internal organ, an abnormal inclusion of an opaque region in an internal organ, an abnormal inclusion of an opaque internal region external to an internal organ (e.g., a shadow between the heart and the lungs), a region of abnormal pigmentation on an external body part, a region of abnormal surface contour of an external body part, an abnormal shape of an internal organ, an absence of an internal organ (e.g., missing appendix), an abnormal size of an internal organ relative to another internal organ, a dislocation of an implanted object, and an absence of a skeletal part (e.g., a missing rib).

A system and a non-transitory computer-readable storage medium also are provided according to the first embodiment. The system may comprise a receiver, a computer processor coupled to a memory, and a transmitter. The receiver may be connected to a communication network, and may be structured to receive a plurality of medical records. Each of the medical records may comprise a digitized image of a body region of a patient. The computer processor may be programmed to, for each of the images: perform a character-recognition process to locate at least one character in the image, the at least one character being one of or a combination of: a symbol character and a text character; mask each of the located at least one character, to obtain a masked image; perform an identification process on the masked image to identify the body region of the image; perform an analysis routine on the masked image to determine a screening score, the analysis routine corresponding to the identified body region; and, if the screening score determined by the analysis routine is within a normal range for the analysis routine, generate a normal notification indicating that the image is a normal image within a healthy range for the identified body region. The transmitter may be connected to the communication network, and may be structured to transmit the normal notification to a requester.

The non-transitory computer-readable storage medium according to the embodiment may store a program that, when executed by a computer, causes the computer to perform the method of the embodiment.

### EMBODIMENT 2

According to a second embodiment of the present technology, a computer-implemented method for screening medical data is provided. The method may comprise electronically obtaining a plurality of medical records provided by a requester. Each of the medical records may comprise an image of a body region of a patient. The method also may comprise utilizing a computer processor to, for each of the images: perform automatically an identification process on the image to identify the body region; select automatically an analysis routine to analyze the identified body region; analyze automatically the medical image using the selected analysis routine to calculate a screening score corresponding to the image; and, if the screening score corresponding to the image is equal to or below a predetermined threshold for the selected analysis routine, generate automatically a normal notification indicating that the image is a normal image within a healthy range for the identified body region.

According to an aspect of the embodiment, the obtaining of the medical records may comprise receiving the medical records transmitted by the requester.

According to another aspect of the embodiment, the obtaining of the medical records may comprise retrieving the medical records from a memory in which the medical records are deposited by the requester.

According to an aspect of the embodiment, the analysis routine may determine the screening score by combining a plurality of sub-scores resulting from a plurality of sub-routines of the analysis routine.

According to an aspect of the embodiment, the method may further comprise automatically transmitting the normal notification to the requester.

According to an aspect of the embodiment, the normal notification may be transmitted to the requester together with other normal notifications generated from the received medical records.

According to an aspect of the embodiment, the utilizing of the computer processor may further comprise, if the screening score corresponding to the image is above the predetermined threshold value for the selected analysis routine, flagging the image for further analysis.

According to an aspect of the embodiment, the utilizing of the computer processor may further comprise removing automatically, from further analysis, each image resulting in a normal notification.

According to an aspect of the embodiment, the utilizing of the computer processor may further comprise obtaining automatically, from a memory accessible by the computer processor, the selected analysis routine.

According to an aspect of the embodiment, the identification process may comprise: identifying at least one character on the image; and masking the at least one character from undergoing analysis by the selected analysis routine.

According to some aspects of the embodiment, the identification process may further comprise performing a character recognition process on the at least one character to determine at least one recognized character.

In an aspect of the embodiment, the identification process may utilize the at least one recognized character to identify the body region.

In another aspect of the embodiment, the at least one character recognized in the character-recognition process may not be utilized in the identification process to identify the body region.

In an aspect of the embodiment, the identification process may utilize the at least one recognized character to identify a type of the image. The type of the image may comprise one of: an x-ray radiographic image, an ultrasound sonographic image, a magnetic-resonance imaging image, an endoscopic photograph, an epidermal photograph, and a nuclear-emission radiographic image.

In an aspect of the embodiment, the identification process may utilize the at least one recognized character to identify the image as any one of or any combination of: a two-dimensional image, a three-dimensional image, a surface image, a cross-sectional image, and a tomographic image in a set of tomographic images.

According to some aspects of the embodiment, the identification process may perform an object-contour routine on the image to identify contours of at least one object in the image.

In an aspect of the embodiment, to identify the body region, the identification process may perform a comparison routine to compare the contours of the at least one object in the image with one or more reference images stored in a memory accessible by the computer processor.

In an aspect of the embodiment, a comparison result of the comparison routine may identify any one of or any combination of: an external body region, an internal body region, an external body part, an internal body part, an internal organ, a foreign object (e.g., an implanted object, a prosthetic device), and a skeletal part.

According to an aspect of the embodiment, each of the medical records may further comprise additional information. The additional information may comprise any one of or any combination of: a previous medical image of the patient, a medical history of the patient, demographic information of the patient, a previous diagnosis of the patient, a physician comment regarding the patient, and an upcoming medical test of the patient. The selected analysis routine may utilize data from the additional information to calculate the screening score corresponding to the image.

According to an aspect of the embodiment, the utilizing of the computer processor may further comprise, prior to the identification process, evaluating the image to determine a quality score for the image. The quality score may take into account any one of or any combination of: a level of blurriness, evidence of patient movement, an appropriateness of magnification, a correctness of imaging view, a presence of a non-patient artifact, an image-digitization artifact, and an inappropriate exposure condition. The utilizing of the computer processor also may comprise, if the quality score for the image is at or above a predetermined threshold value, generating a rejection notification indicating that the image was rejected for being of insufficient quality to enable a reliable analysis; and, if the quality score for the image is below the predetermined threshold value, proceeding to perform the identification process.

A system and a non-transitory computer-readable storage medium also are provided according to the second embodiment. The system may comprise a receiver, transmitter, and a computer processor coupled to a memory. The receiver may be connected to a communication network, and may be structured to receive a plurality of medical records. Each of the medical records may comprise an image of a body region of a patient. The computer processor may be programmed to, for each of the images: perform an identification process on the image to identify the body region; select an analysis routine to analyze the identified body region; analyze the medical image using the selected analysis routine to calculate a screening score corresponding to the image; and, if the screening score corresponding to the image is equal to or below a predetermined threshold for the selected analysis routine, generate a normal notification indicating that the image is a normal image within a healthy range for the identified body region. The transmitter may be connected to the communication network, and may be structured to transmit the normal notification to a requester.

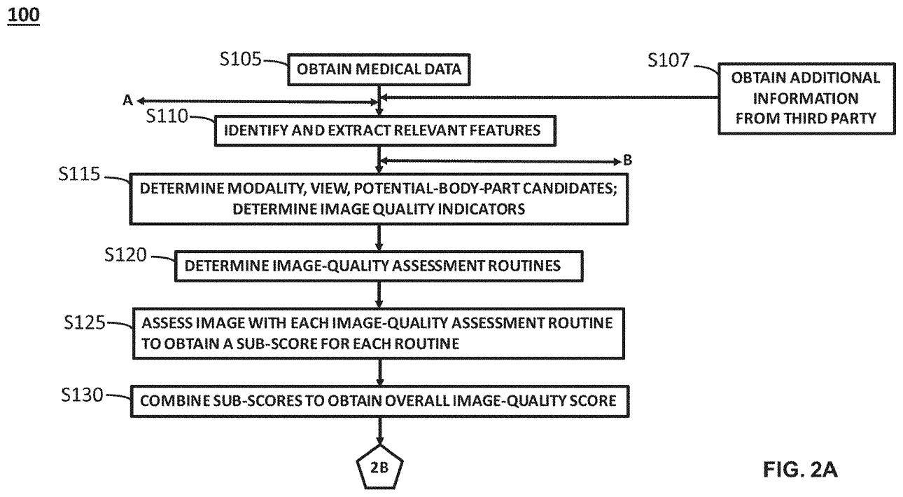

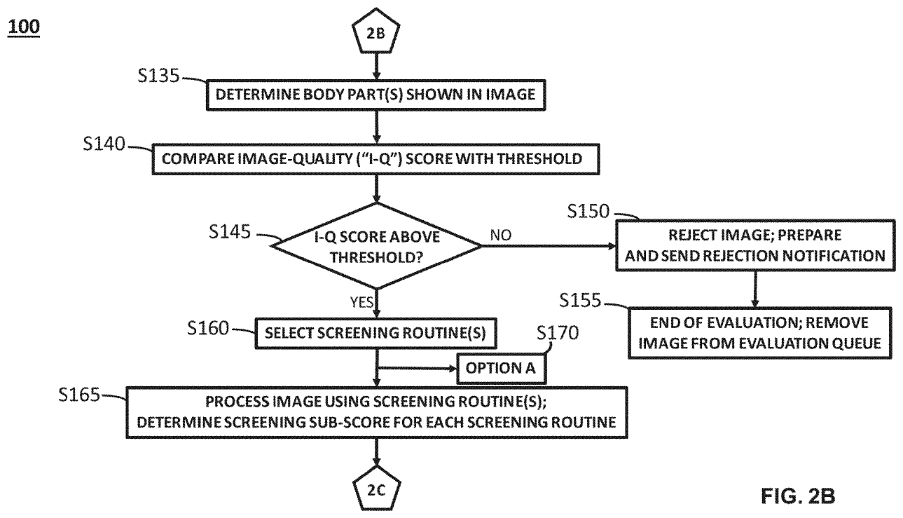

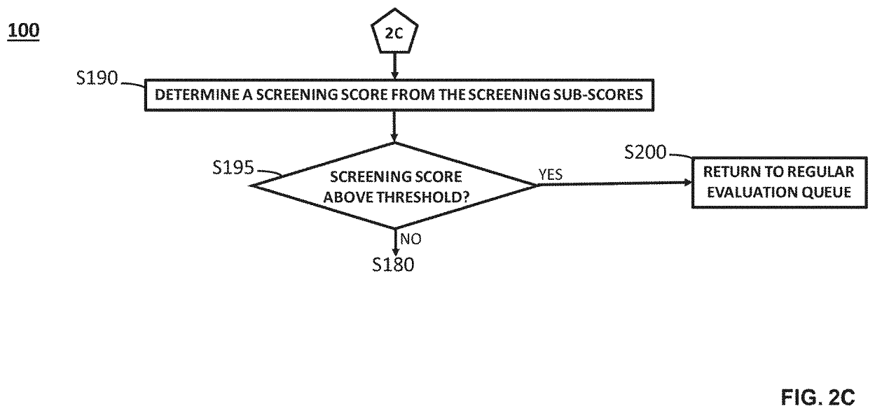

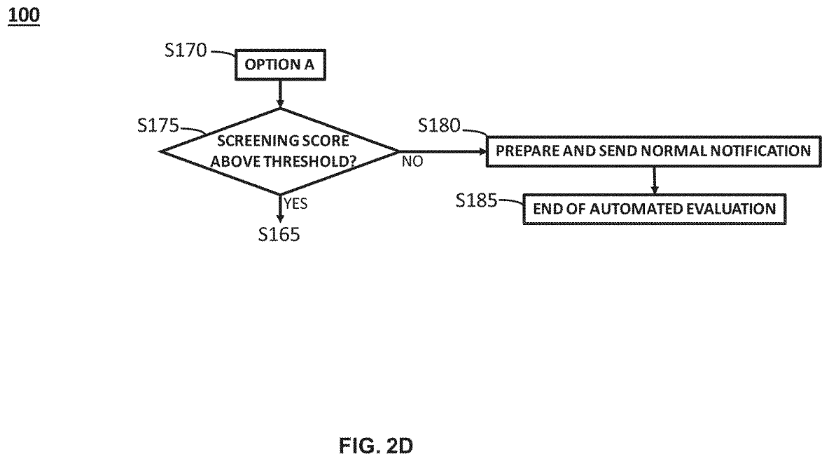

Turning to the figures, FIGS. 2A-2D show flowcharts for a workflow 100 of a computer system of a medical evaluation facility, according to an aspect of the present technology. In FIG. 2A, at S105, medical data from a requester is obtained by the system. The medical data may include one or more images from one or more imaging studies. The medical data also may include additional data from the requester, such as the additional data described above.

Optionally, at S107, the medical evaluation facility may receive additional information from a third-party source other than the requester. For example, PCPs and/or health-insurance providers may provide information directly to the medical evaluation facility.

At S110, each image and the additional information provided for that image, if any, are processed to extract or identify relevant features of the image. Selected data extracted from the additional information may be utilized to extract or identify the relevant features from the image.

The relevant features may include any combination of: a change in contrast in one or more regions of the image, indicating an object; a location of the object(s) relative to borders of the image; a contour representing a periphery or border of each object, and a width of the contour; an area of the object(s). The relevant features also may include items of information extracted from the additional information provided for the image. As will be appreciated, the relevant features may include other features not specifically listed above.

At S115, some or all of the relevant features extracted or identified at S110 may be used to determine a modality of the image, a view of the image, and potential-body-part candidates.

The modality may be one of the following: x-ray radiographic image (XR), nuclear-emission radiographic image (NE), ultrasound sonographic image (US), magnetic-resonance imaging image (MRI), endoscopic photograph (ENP), epidermal photograph (EPP), two-dimensional image (2D), three-dimensional image (3D), surface image (SF), cross-sectional image (CS), tomographic image (CT). As will be appreciated, this list of modalities is not exhaustive, and other modalities and sub-modalities are possible.

The potential-body-part candidates may be determined from any one of or any combination of: the additional information provided in the medical data from the requester; the third-party additional information provided for the image, if any; and via computer-vision image-processing techniques for identifying objects and/or shapes in images.

Also, at S115, some or all of the set of features extracted or identified at S110 may be used to determine image-quality indicators for the image. The image-quality indicators may include indicators for any one of or any combination of: a level of blurriness, evidence of patient movement, an appropriateness of magnification, a correctness of imaging view, a presence of a non-patient artifact, a presence of an image-digitization artifact, and an overexposure or underexposure condition. As will be appreciated, other indicators of image quality, not specifically mentioned above, also may be included.

At S120, based on the image-quality indicators determined at S115, one or more image-quality assessment routines (algorithms) may be selected from a database of image-quality routines stored in a memory accessible by the system. At S125, the image may undergo processing by the selected one or more image-quality assessment routines to determine a sub-score for each of the image-quality indicators. At S130, the sub-scores may be combined to obtain an overall image-quality score.

For example, if a level of blurriness and a presence of an image-digitization artifact are included in the set of image-quality indicators, a routine for evaluating blurriness in an image and a routine for evaluating digitization artifacts in an image may be selected from the database. Each routine may be used to process the image and determine a score. The blurriness routine may, for example, return a result indicating that a lower-right corner of the image is slightly blurry, amounting to about 5% of the total number of pixels of the image. The blurriness routine may, for example, return a score of 0.5 out of 10, indicating a very low risk for the blurriness to prevent a reliable medical assessment of the image. The digitization-artifact routine may, for example, return a result indicating that a row of pixels was corrupted in the digitization process, and the corrupted row of pixels runs through a region within 2.5 cm from a top border of the image. The digitization-artifact routine may, for example, return a score of 1.1 out of 10, indicating a low but appreciable risk of the corrupted pixels preventing a reliable medical assessment of the image. The combined score of 1.6 may be determined as the image-quality score for the image.

In another example, if an improper exposure condition (e.g., overexposure or underexposure) is included in the set of image-quality indicators, a routine for evaluating exposure may be selected from the database. The exposure routine may, for example, return a result indicating that over 90% of the pixels have a brightness exceeding 85 out of a maximum brightness of 100, with an average brightness of 80, a minimum brightness of 70, and a maximum brightness of 100. The exposure routine may, for example, return a score of 8.0 out of 10, indicating a high risk of overexposure or excessive brightness preventing a reliable medical assessment of the image. If no other score is returned for the set, the score of 8.0 may be determined as the image-quality score for the image.

At S135, in FIG. 2B, based on the potential-body-part candidates determined at S115, one or more body-part determination routines may be selected from a database of body-part determination routines stored in a memory accessible by the system. The image may undergo processing by the selected one or more body-part determination routines to determine a score for each of the potential-body-part candidates. For example, each routine may correspond to a potential body part, and a score may be determined for each routine. The scores may be compared to determine the body part(s) of the image. For example, if only one score exceeds 5 out of 10, the body part of the image corresponds to the body part of the routine resulting in the over 5 score. If more than one score exceeds 5, the image may show more than one body part, with the body parts of the image corresponding to the body parts of the routines resulting in the over 5 score.

Optionally, the additional information provided with the image may include an indication of the body part(s) of the image. In such a case, body-part determination using a selected body-part determination routine(s) may be omitted and the body part(s) indicated in the additional information may be associated with the image, or, alternatively, may be performed to confirm the body part(s) indicated in the additional information.

At S140, the image-quality score for the image may be compared with a predetermined image-quality threshold for the modality determined for the image and the body-part of the image. At S145, if the image-quality score is at or below the predetermined image-quality threshold, indicating that the image quality is sufficient to enable a reliable medical assessment, then further evaluation of the image is permitted. In the above example in which the image-quality score of 1.6 was determined, if the image is an x-ray image (i.e., XR modality) of a finger, and the predetermined image-quality threshold for an x-ray finger image is 2.5, then the image may be allowed to continue for further evaluation. However, if the image is an x-ray image of a heart, and the predetermined image-quality threshold for an x-ray heart image is 1.5, then the image may not be allowed to continue but instead may be rejected from further evaluation.

At S145, if the image-quality score is above the predetermined image-quality threshold, indicating that the image quality is insufficient to enable a reliable medical assessment, then, at S150, the image is rejected from further evaluation and a rejection notification is automatically generated by the system and transmitted to the requester. In the above example in which the image-quality score of 8.0 was determined, the image may be rejected from further evaluation regardless of the modality of the image, if none of the predetermined image-quality thresholds for the various possible modalities has a value of 8 or above. At S155, the rejected image is removed from the medical evaluation facility's valuation queue.

If the image is permitted to continue for further evaluation, at S160, a further screening or evaluation of the image utilizes the modality of the image, the view of the image (e.g., an AP (anteroposterior) view, in which radiation is incident on the patient's front side and the radiation film is proximate the patient's back; a PA (posteroanterior) view, in which radiation is incident on the patient's back side and the radiation film is proximate the patient's front; etc.), the body part(s) of the image, and, optionally, the relevant features of the image extracted or identified at S110, as parameters for selecting one or more screening routines. That is, the parameters may be used in a selection algorithm for one or more sets of screening routines from a database of screening routines stored in a memory accessible by the processor.

At S165, the image is evaluated using the one or more sets of screening routines to determine whether any abnormality or unusual feature is present in the image.

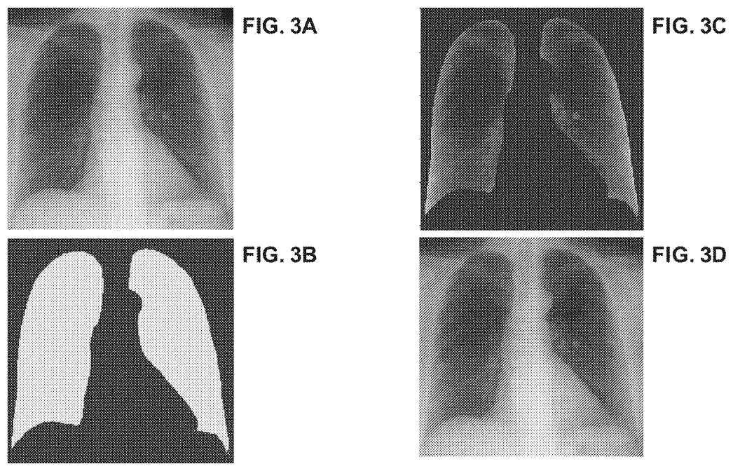

For example, if the image is determined to be an x-ray image of a pair of lungs (i.e., both right and left lungs), such as shown in FIG. 3A, with the image being taken from the patient's back side (i.e., the x-rays were directed at the patient's back side in the imaging processing), then the selection algorithm may select a set of one or more screening routines that may enhance the contrast of the image and/or use known computer-vision techniques to evaluate pixels of the image to recognize boundaries or borders of each of the lungs, such as shown in FIG. 3B. The one or more screening routines also may use the recognized boundaries of each of the lungs to extract the imaged lung regions from the image (or, conversely, to block out all areas outside of the recognized boundaries) to produce an extracted image, such as shown in FIG. 3C. At S165, the one or more screening routines may be used to process the extracted image to determine whether an abnormality or unusual feature is present.

Optionally, prior to S165, at S170 (“OPTION A”), one or more initial screening routines may be used to compare the extracted image with reference images for healthy lungs, to find evidence of differences, as shown in FIG. 2D. If little or no such evidence is found, an initial screening sub-score of 0.5 out of 10 may be determined for these one or more screening routines. At S175, a determination is made as to whether the initial screening sub-score is above a predetermined threshold for the initial screening routine(s) for lungs. The predetermined threshold may be such that a value at or below the predetermined threshold is an indication of a high degree of certainty for a healthy body lung. At S180, if the initial screening sub-score is at or below the predetermined threshold, the system automatically generates a normal notification and sends the normal notification to the requester. At S185, automated evaluation of the image may end, and the image may be removed from the medical evaluation facility's evaluation queue. Optionally, before the normal notification is sent and before the image is removed from the evaluation queue, the image may be reviewed by a non-specialist clinician (e.g., a trained nurse). If the non-specialist clinician confirms that the image shows no signs of an abnormality or an unusual feature, then the normal notification may be sent to the requester and the image may be removed from the queue.

At S175, if the initial screening sub-score is above the predetermined threshold, then the workflow may proceed to S165 to continue evaluating the extracted image with screening routines.

One or more screening routines may be used to scan the extracted image for evidence of shadows or graded contrast differences. If no such evidence is found, a screening sub-score of 0 may be determined for these one or more screening routines. If such evidence is found, the workflow proceeds to continue the evaluation of the extracted image. Depending on the evidence found, one or more screening routines may be selected to compare the extracted image with reference images for lungs showing emphysema as well as reference images for lungs shown pneumonia. A screening sub-score may be determined based on similarities found between the extracted image and the reference images.

One or more screening routines may be used to scan the extracted image for evidence of a region or regions in which there is a sharp change in contrast. If no such evidence is found, a screening sub-score of 0 may be determined for these one or more screening routines. If such evidence is found, such as in FIG. 3D, the workflow proceeds to continue the evaluation of the extracted image. Depending on the evidence found, one or more screening routines may be selected to compare the extracted image with reference images for tumors in lungs. A screening sub-score may be determined based on similarities found between the region(s) of the extracted image and the reference images.

One or more screening routines may be used to scan the extracted image for texture characteristics, such as surface texture of a region of the body part for evidence of an abnormal texture or an abnormal change in texture. Such screening may be used for determining regions of skin cancer or regions having pre-cancerous texture. FIG. 12 shows examples of different types of textures that may be evaluated by a texture-screening routine. As will be appreciated, other types of textures, not shown in FIG. 12, also may be evaluated by a texture-screening routine.

As will be appreciated, a battery of different sets of one or more screening routines may be used to evaluate the extracted image, and each set may result in a screening sub-score.

At S190, when the screening for abnormalities and unusual features has been completed, a screening score may be determined by combining all the screening sub-scores (e.g., by simple addition, a weighted sum, or the like). At S195, if the screening score is above a predetermined screening threshold, indicating that there is at least one abnormality or unusual feature in the extracted image that requires further evaluation, then, at S200, the image is returned to a regular standard evaluation queue for further machine-based evaluation or further evaluation by trained personnel. However, if the screening score is at or below the predetermined screening threshold, indicating with a high degree of certainty that no abnormality or unusual feature was found, then, at S180, a normal notification is automatically generated and sent to the requester.

The normal notification may indicate that the image was evaluated and the body region of the image showed no signs of an abnormality of unusual feature, indicating a healthy body region (e.g., a healthy lung, a healthy stomach, a healthy abdomen, a healthy brain, a healthy bone region, etc.). The notification may include a summary of the screening routines performed and the image-quality indicators evaluated, and may further include scores and sub-scores for the screening routines and/or the image-quality indicators.

In the workflow 100 described above, the image-quality evaluation procedure is performed before the abnormality screening procedure. As will be appreciated, although not shown in FIGS. 2A-2D, these procedures may be performed in an opposite order, or may be performed concurrently.

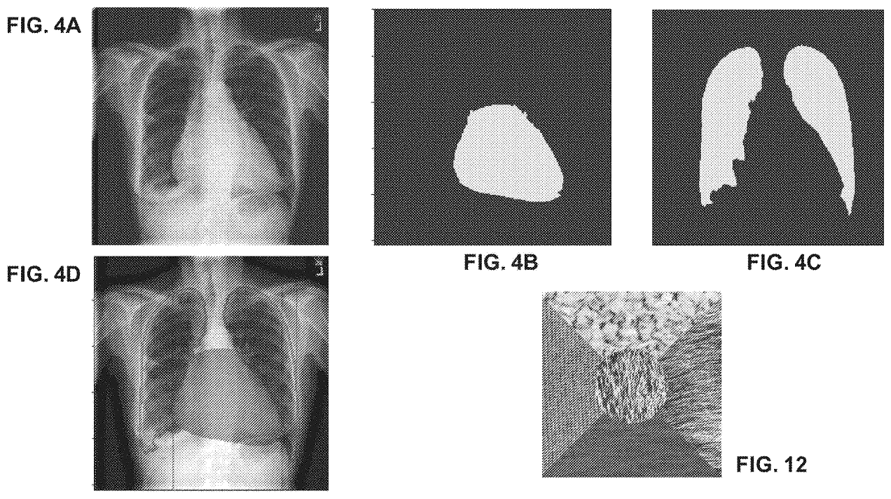

As mentioned above, the image may be determined to show more than one body part. For example, the image may show a pair of lungs as well as a heart, such as shown in FIG. 4A, with the image showing a PA view. In such a case, the selection algorithm may select a set of one or more screening routines that may: enhance the contrast of the image; use known computer-vision techniques to recognize boundaries or borders of the heart, such as shown in FIG. 4B; and use known computer-vision techniques to recognize boundaries or borders of each of the lungs, such as shown in FIG. 4C. The one or more screening routines also may use the recognized boundaries of the heart and each of the lungs to extract the imaged heart and lung regions from the image. The location and dimensions of the heart relative to the location and dimensions of the lungs may be compared with the same dimensions of one or more reference images showing lungs and a heart in a PA view, to determine whether the relative positioning and/or the relative sizes of the heart and lungs in the image is within a predetermined range of the relative positioning and/or the relative sizes of the heart and lungs in, for example, a reference image of a healthy patient. For example, any one of or any combination of the following quantities may be measured and compared with one or more reference images: a maximum length of the heart relative to a maximum length of the lungs; a maximum width of the heart relative to a maximum width of the lungs; a two-dimensional area (i.e., an area determined from the two-dimensional image) of the image of the heart relative to a two-dimensional area of the lungs; etc. FIG. 4D shows an example of such a measurement.

In FIG. 4D, the imaged heart is shown in a masked view (see FIG. 4B) relative to the lungs of the image, with the boundaries of the lungs outlined in the image. Width lines are overlayed on the image in FIG. 4D to show how the one or more screening routines may determine the width of the heart and the overall width of the lungs, and calculate a ratio of the overall width of the lungs W_{L} to the width of the heart W_{H}. The ratio W_{L}/W_{H} of the image may be compared with values in a lookup table stored in a memory and accessible by the system. If the ratio is within a first range of a target ratio then the relative sizes of the heart and the lungs may be determined to be normal, and a screening sub-score in the range, e.g., 0.0 to 1.0 may be determined, depending on the deviation from the target ratio. If the ratio is in a second range, above the first range, then the relative sizes of the heart and the lungs are determined to be abnormal, and the heart may be designated as oversized. If the ratio is in a third range, below the first range, then the relative sizes of the heart and the lungs are determined to be abnormal, and the heart may be designated as undersized. For an oversized or undersized heart, a screening sub-score in the range of 10.0 may be determined, which may be a value sufficiently high to ensure that the image will undergo further evaluation by trained personnel.

If the additional information provided with the image does not indicate a gender for the patient of the image, the image may be processed to determine the patient's gender based on features in the image that may appear differently for the different genders. Knowledge of the patient's gender may be important for determining appropriate screening routines for evaluating the image.

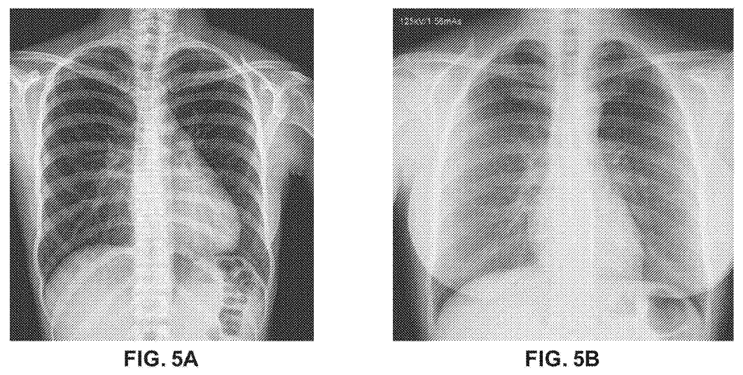

FIG. 5A shows an x-ray image of a healthy male, and FIG. 5B shows an x-ray image of a healthy female. One or more screening routines may be used to determine gender based on, for example, bone structure and/or the chest shape/size relative to other structures in the image, etc. As evident in FIGS. 5A and 5B, the size of the lungs may differ between a male and a female, and the size and location of the heart may differ between a male and a female. Therefore, for some medical evaluations, knowledge of gender may be important, if not required, for a reliable diagnosis to be possible. In such cases, the selection algorithm may select a set of one or more screening routines that may determine gender with a high degree of certainty. Similar to one or more of the routines discussed above, the image may be processed with a gender screening routine to determine a value for a parameter indicative of a female (for example), and may compare this parameter to a predetermined threshold for the gender screening routine. If the value is above the predetermined threshold, it may be determined with a high degree of certainty that the patient of the image is a female. On the other hand, if the value is at or below the predetermined threshold, it may be determined that one or more further screenings need to be performed before it can be determined with a high degree of certainty that the patient of the image is a male, or before it can be determined that the gender of the patient cannot be determined with a high degree of certainty from the screening routines available.

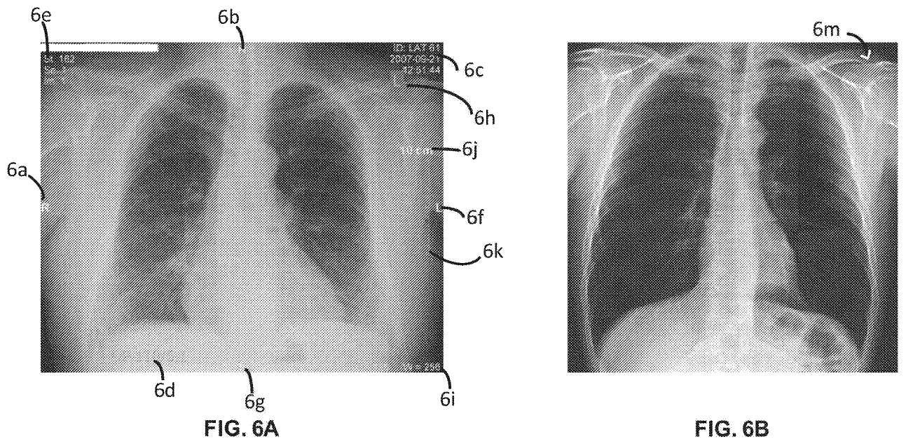

Medical images may contain characters and graphics, which may be useful for providing information to trained personnel. The characters be, e.g., symbols and/or alphanumeric text, and the graphics may be, e.g., a scale or ruler, border lines, and/or any type of illustration. For example, FIGS. 6A shows an x-ray image of an upper torso of a patient. The image includes alphanumeric text 6a-6j and graphics 6k. Characters and graphics on the image may provide information to trained personnel evaluating the image. For example, the “R” at 6a may indicate the patient's right side, the “H” at 6b may indicate a direction to the patient's head, the text at 6i may indicate a length and a width relevant to the image, and the graphics at 6k may indicate a scale for determine the size of features of the image. In FIG. 6B, a symbol 6m appears near the top right corner, which may be a check mark or a reversed “L” at an oblique angle.

Although potentially useful for trained personnel, characters and graphics (collectively referred to as “text”) may interfere with machined-based image processing, which may evaluate image features on a pixel scale. Accordingly, removal or masking of text may be desirable before the image is processed to determine whether an abnormality or unusual feature is present.

An aspect of the present technology may utilize known character-recognition and graphics-object detection algorithms to identify the presence of text and/or graphics. These algorithms or another algorithm may be used to hide or mask the identified text and/or graphics, such as, e.g., by flagging all pixels corresponding to the identified text and/or graphics and bypassing the flagged pixels when the image undergoes subsequent processing to detect may be flagged by these algorithms. As will be appreciated, other ways besides flagging may be used to prevent pixels corresponding to text and/or graphics from undergoing subsequent processing.

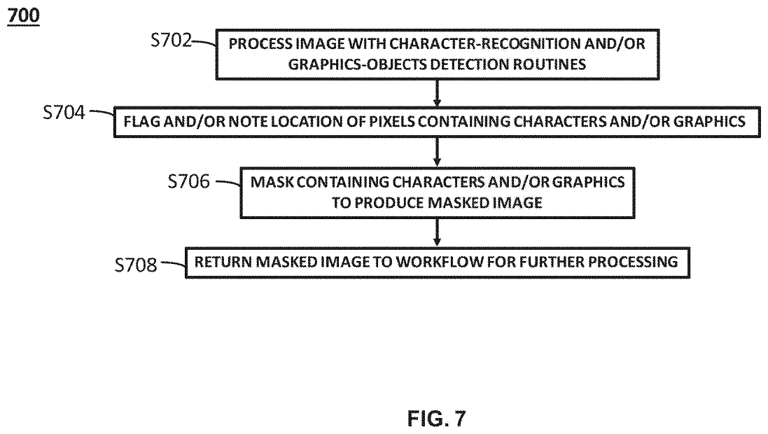

FIG. 7 shows a flow chart for a routine 700 that may be used to mask text and/or graphics in the image. The routine 700 may be performed at point A or B of the workflow 100 (FIG. 2A), performing the routine at other points of the workflow 100 may be possible.

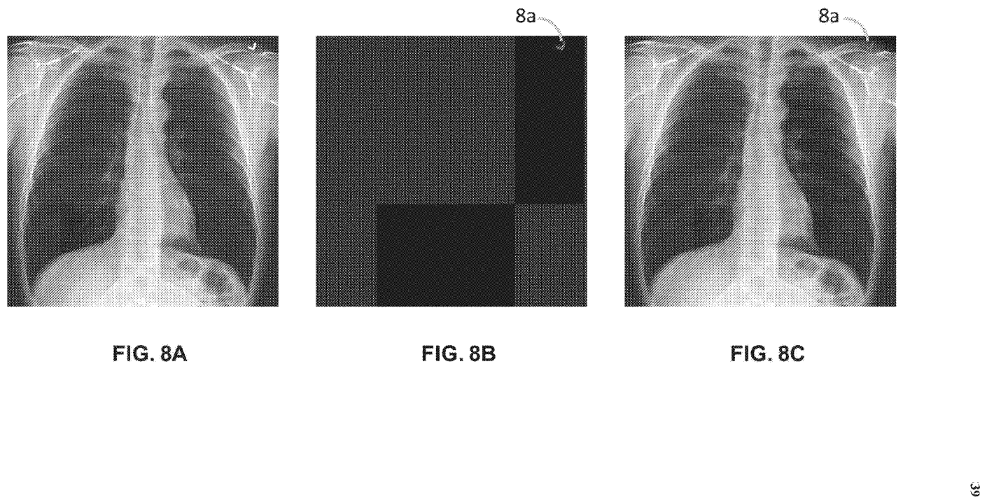

At S702, the image is processed using one or more character-recognition and graphics-object detection routines to identify the presence of alphanumeric character(s) and/or graphics. FIG. 8A shows an example of an image before processing using one or more character-recognition and graphics-object detection routines. At S704, pixels corresponding to the identified characters and/or graphics (“character/graphics pixels”) are flagged and/or their positions are noted. FIG. 8B shows a symbol 8a identified by the one or more character-recognition and graphics-object detection routines. At S706, the character/graphics pixels are masked to produce a masked image, shown in FIG. 8C. In the masked image of FIG. 8C, the identified symbol is shown to be shaded or colorized to indicate that the pixels of the identified symbol have has been flagged and masked, such that these pixels will not be considered when the masked image undergoes processing to determine the presence of an abnormality or unusual feature, if any, in the masked image. At S708, the masked image may be returned to the workflow 100 for further processing.

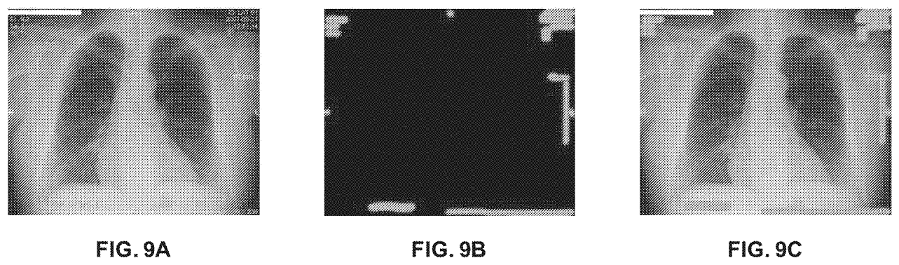

Alternatively, FIG. 9A shows an example of an image before processing using one or more character-recognition and graphics-object detection routines. FIG. 9C shows regions of text and/or graphics identified by the one or more character-recognition and graphics-object detection routines. Unlike FIG. 8B, in FIG. 9B a region around an identified character (e.g., the “H” in FIG. 9A) may be designated for masking, a region around an identified graphics object (e.g., the scale in FIG. 9A) may be designated for masking, and a region around a string of alphanumeric characters may be designed for masking. FIG. 9C shows a masked image, in which the regions containing identified text and/or graphics are shown to be shaded or colorized to indicate that the pixels of the region have been masked and will not be considered when the masked image undergoes processing to determine the presence of an abnormality or unusual feature, if any, in the masked image. The masked image may be returned to the workflow 100 for further processing.

Aspects of the present technology may be implemented using hardware, software, or a combination thereof, and may be implemented in one or more computer systems or other processing systems. Useful machines for performing some or all of the operations described herein may include digital computers systems, which may be coupled to one or more communication networks.

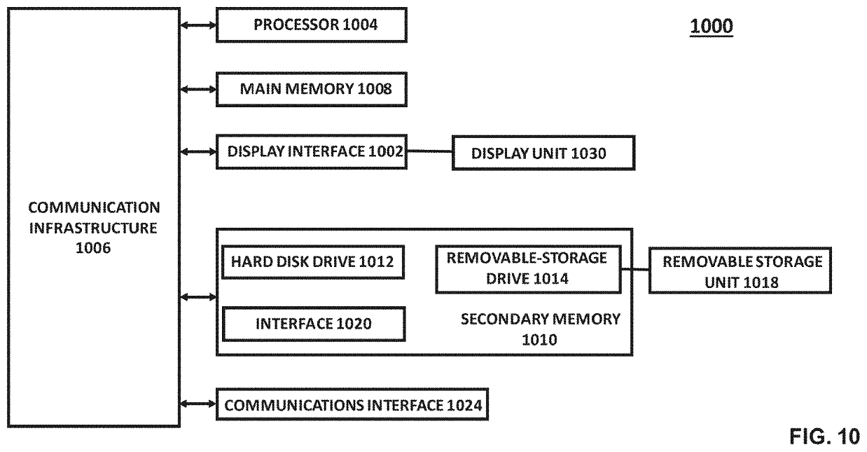

An example of a computer system 1000 that may be utilized for aspects of the present technology is shown in FIG. 10. The computer system 1000 may include at least one processor 1004. Each processor 1004 may be connected to a communication infrastructure 1006 (e.g., a communications bus, a local-area network, and the like). The computer system 1000 may include a display interface 1002 that forwards graphics, text, and other data from the communication infrastructure 1006 for display on a display unit 1030. The computer system 1000 also may include a main memory 1008, which may be a random access memory (RAM), and may also include a secondary memory 1010, which may be a hard disk drive 1012 and/or a removable-storage drive 1014. The removable-storage drive 1014 may read from and/or write to a removable storage unit 1018 in a well-known manner. The removable storage unit 1018 may be, for example, an optical disk, a memory stick, and the like, which may be written to and read by the removable-storage drive 1014. An interface 1020 may be included to allow software and data to be transferred from a removable storage unit 1022 to the computer system 1000. As will be appreciated, the removable storage unit 1018 may be a non-transitory computer-readable storage medium having stored therein data and/or software executable by one or more of the at least one processor 1004. The computer system 1000 also may include a communications interface 1024, which may allow software and/or data to be transferred between the computer system 1000 and external devices (not shown in FIG. 10). For example, the removable storage unit 1018 may be a non-transitory computer-readable storage medium having stored therein program code that, when executed by the at least one processor 1004, causes the at least one processor to perform one or more of the routines and functions, including control functions, described above. Accordingly, the program code may represent a controller of the computer system 1000.

As mentioned above, the present technology may be implemented using hardware and/or software. In the case of a hardware implementation or a hardware and software implementation, hardware components such as application-specific integrated circuits (“ASICs”) may be used. Arrangements of hardware components to perform the functions described herein will be apparent to persons skilled in the relevant art(s).

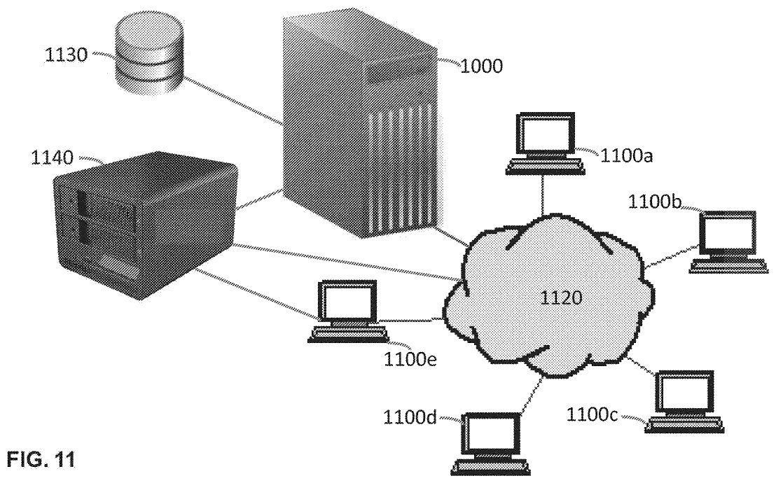

FIG. 11 schematically shows an arrangement for one or more requesters to communicate with the computer system described above. The computer system 1000 may communicate with requester computers 1100a-1100e via a communication network 1120. A database 1130 and/or an external memory system 1140 may be accessible by the computer system 1000 to store information therein or retrieve information therefrom. The communication network 1120 may be the Internet or any other means of communication between the computer system 1120 and the requester computers 1100a-1100e, whether wired or wireless. The requester computers 1100a-1100e may be any computer or digital device able to perform data communication with the communication system 1000. The database 1130 and/or the external memory system may communicate with the computer system 1000 via the communication network 1120 or directly via a dedicated communication channel. Similarly, the external memory system may communicate with the requester computers 1100a-1100e via the communication network 1120 or directly via a dedicated communication channel. Thus, the requester computers 1100a-1100e may provide medical data to the computer system 1000 via the communication network 1120, or the medical data may be uploaded to the external memory system 1140 for retrieval by the computer system 1000.

U.S. Provisional Application No. 62/670,119, filed in the U.S. Patent and Trademark Office on May 11, 2018, is incorporated by reference herein.

It should be understood that various alterations, modifications, and improvements may be made to the structures, configurations, and methods discussed above, and are intended to be within the spirit and scope of the invention disclosed herein. Further, although advantages of the present invention are indicated, it should be appreciated that not every embodiment of the invention will include every described advantage. Some embodiments may not implement any features described as advantageous herein. Accordingly, the foregoing description and attached drawings are by way of example only.

It should be understood that some aspects of the present technology may be embodied as one or more methods, and acts performed as part of a method of the present technology may be ordered in any suitable way. Accordingly, embodiments may be constructed in which acts are performed in an order different than shown and/or described, which may include performing some acts simultaneously, even though shown and/or described as sequential acts in various embodiments.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the description and the claims to modify an element does not by itself connote any priority, precedence, or order of one element over another, or the temporal order in which acts of a method are performed, but are used merely as labels to distinguish one element or act having a certain name from another element or act having a same name (but for use of the ordinal term) to distinguish the elements or acts.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean either of the elements conjoined by the phrase, or both of the elements conjoined by the phrase.

As described, some aspects of the present technology may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A method for expediting screening of medical data, comprising:
electronically obtaining a plurality of medical records from a requester, each of the medical records comprising a digitized image of a body region of a patient;
for each of the images:
performing, using a computer processor, a character-recognition process to locate at least one character in the image, the at least one character being one of or a combination of: a symbol character and a text character;
masking, using a computer processor, each of the located at least one character, to obtain a masked image;
performing, using a computer processor, an identification process on the masked image to identify the body region of the image;
performing, using the computer processor, an analysis routine on the masked image to determine a screening score, the analysis routine corresponding to the identified body region; and,
if the screening score determined by the analysis routine is within a normal range for the analysis routine, generating, using the computer processor, a normal notification indicating that the image is a normal image within a healthy range for the identified body region; and
automatically transmitting the normal notification to the requester.

2. The method of claim 1, wherein the obtaining of the medical records comprises receiving the medical records transmitted by the requester.

3. The method of claim 1, wherein the obtaining of the medical records comprises retrieving the medical records from a memory in which the medical records are deposited by the requester.

4. The method of claim 1, wherein the analysis routine determines the screening score by combining a plurality of sub-scores resulting from a plurality of sub-routines of the analysis routine.

5. The method of claim 1, wherein the normal notification is transmitted to the requester together with other normal notifications generated from the received medical records.

6. The method of claim 1, wherein the performing of the character-recognition process to locate the at least one character in the image recognizes the at least one character in the image.

7. The method of claim 6, wherein the character-recognition process recognizes the at least one character in the image to be alphanumeric text corresponding to the image.

8. The method of claim 7, wherein the alphanumeric text is a word or a string of words corresponding to the image.

9. The method of claim 6, wherein the analysis routine comprises:
identifying indicator information,
obtaining first and second information from the indicator information,
processing the image using the first information to determine a first factor,
processing the image using the second information to determine a second factor, and
calculating the screening score using at least the first and second factors.

10. The method of claim 9,
wherein the first information of the indicator information comprises image-quality information, and
wherein the second information of the indicator information comprises the body region identified in the identification process.

11. The method of claim 10, wherein the at least one character recognized in the character-recognition process is utilized in the identification process to identify the body region of the image.

12. The method of claim 10, wherein the at least one character recognized in the character-recognition process is not utilized in the identification process to identify the body region of the image.

13. The method of claim 10, wherein the identification process performs an object-contour routine on the image to identify contours of at least one object in the image.

14. The method of claim 13, wherein, to identify the body region, the identification process performs a comparison routine to compare the contours or a portion of the contours of the at least one object in the image with one or more reference images stored in a memory accessible by the computer processor.

15. The method of claim 9,
wherein the first factor is determined by comparing the first information to first reference information stored in a database accessible by the computer processor, and
wherein the second factor is determined by comparing the second information to second reference information stored in a database accessible by the computer processor.

16. The method of claim 10, further comprising:
evaluating the image to determine the image-quality information, the image-quality information comprising a quality value for any one of or any combination of:
a level of blurriness,
evidence of patient movement,
an appropriateness of magnification,
a correctness of imaging view,
a presence of a non-patient artifact,
an image-digitization artifact,
an overexposure condition, and
an underexposure condition.

17. The method of claim 1,
wherein each of the medical records further comprises additional information, the additional information comprising any one of or any combination of:
a related previous medical image of the patient,
a medical history of the patient,
demographic information of the patient,
a previous diagnosis of the patient,
a physician comment regarding the patient, and
an upcoming medical test of the patient, and
wherein the analysis routine takes into account the additional information to determine the screening score.

18. The method of claim 10, wherein the second information comprises a type of the image, the type of the image comprising one of:
an x-ray radiographic image,
an ultrasound sonographic image,
a magnetic-resonance imaging image,
an endoscopic photograph,
an epidermal photograph, and
a nuclear emission radiographic image.

19. The method of claim 10, wherein the second information comprises a category of the image, the category comprising any one of or any combination of:
a two-dimensional image,
a three-dimensional image,
a surface image,
a cross-sectional image, and
a tomographic image in a set of tomographic images.

20. The method of claim 19, wherein the second information is obtained from the image or from additional information obtained by the computer processor separately from the medial records.

21. The method of claim 15,
wherein the processing using the first information determines an image-quality score for the first factor, and compares the image-quality score to a threshold value above which a reliable analysis cannot be made from the image, and
wherein the method further comprises generating and transmitting to the requester a rejection notification indicating that the image corresponding to the image-quality score was rejected from analysis due to low image quality, when the image-quality score is above the threshold value.

22. The method of claim 21, wherein the processing using the second information commences after the image-quality score is determined to be at or below the threshold value.

23. The method of claim 15, wherein the processing using the second information comprises determining the image to be any one or any combination of:
an external body region,
an internal body region,
an external body part,
an internal body part,
an internal organ,
an implanted object,
a prosthetic device, and
a skeletal part.

24. The method of claim 15, wherein the processing using the second information comprises determining whether the image includes an anomaly.

25. The method of claim 24, wherein the anomaly comprises any one of or any combination of:
a bone fracture,
a joint dislocation,
an abnormal surface contour of an internal organ,
an abnormal surface texture of an external body part,
an abnormal surface texture of an internal organ,
an abnormal inclusion of an opaque region in an internal organ,
an abnormal inclusion of an opaque internal region external to an internal organ,
a region of abnormal pigmentation on an external body part,
a region of abnormal surface contour of an external body part,
an abnormal shape of an internal organ,
an absence of an internal organ,
an abnormal size of an internal organ relative to another internal organ,
a dislocation of an implanted object, and
an absence of a skeletal part.

26. A system for expediting screening of medical data, comprising:
a receiver connected to a communication network, the receiver structured to receive a plurality of medical records, each of the medical records comprising a digitized image of a body region of a patient;
a computer processor coupled to a memory, the computer processor programmed to, for each of the images:
perform a character-recognition process to locate at least one character in the image, the at least one character being one of or a combination of: a symbol character and a text character,
mask each of the located at least one character, to obtain a masked image,
perform an identification process on the masked image to identify the body region of the image,
performing an analysis routine on the masked image to determine a screening score, the analysis routine corresponding to the identified body region, and,
if the screening score determined by the analysis routine is within a normal range for the analysis routine, generate a normal notification indicating that the image is a normal image within a healthy range for the identified body region; and
a transmitter connected to the communication network, the transmitter structured to transmit the normal notification to a requester.

27. The system claim 26, wherein the receiver receives the medical records transmitted by the requester.

28. The system of claim 26, wherein the receiver retrieves the medical records from a memory in which the medical records are deposited by the requester.

29. The system of claim 26, wherein the analysis routine determines the screening score by combining a plurality of sub-scores resulting from a plurality of sub-routines of the analysis routine.

30. The system of claim 26, wherein the transmitter transmits the normal notification to the requester together with other normal notifications generated from the medical records.

31. The system of claim 26, wherein the character-recognition process performed to locate the at least one character in the image recognizes the at least one character in the image.

32. The system of claim 31, wherein the character-recognition process recognizes the at least one character in the image to be alphanumeric text corresponding to the image.

33. The system of claim 32, wherein the alphanumeric text is a word or a string of words corresponding to the image.

34. The system of claim 31, wherein the analysis routine comprises:
identifying indicator information,
obtaining first and second information from the indicator information,
processing the image using the first information to determine a first factor,
processing the image using the second information to determine a second factor, and
calculating the screening score using at least the first and second factors.

35. The system of claim 34,
wherein the first information of the indicator information comprises image-quality information, and
wherein the second information of the indicator information comprises the body region identified in the identification process.

36. The system of claim 35, wherein the at least one character recognized in the character-recognition process is utilized in the identification process to identify the body region of the image.

37. The system of claim 35, wherein the at least one character recognized in the character-recognition process is not utilized in the identification process to identify the body region of the image.

38. The system of claim 35, wherein the identification process performs an object-contour routine on the image to identify contours of at least one object in the image.

39. The system of claim 38, wherein, to identify the body region, the identification process performs a comparison routine to compare the contours or a portion of the contours of the at least one object in the image with one or more reference images stored in a memory accessible by the computer processor.

40. The system of claim 34,
wherein the first factor is determined by comparing the first information to first reference information stored in a database accessible by the computer processor, and
wherein the second factor is determined by comparing the second information to second reference information stored in a database accessible by the computer processor.

41. The system of claim 35, wherein the computer processor is programmed to, for each of the images, evaluate the image to determine the image-quality information, the image-quality information comprising a quality value for any one of or any combination of:
a level of blurriness,
evidence of patient movement,
an appropriateness of magnification,
a correctness of imaging view,
a presence of a non-patient artifact,
an image-digitization artifact,
an overexposure condition, and
an underexposure condition.

42. The system of claim 26,
wherein each of the medical records further comprises additional information, the additional information comprising any one of or any combination of:
a related previous medical image of the patient,
a medical history of the patient,
demographic information of the patient,
a previous diagnosis of the patient,
a physician comment regarding the patient, and
an upcoming medical test of the patient, and
wherein the analysis routine takes into account the additional information to determine the screening score.

43. The system of claim 35, wherein the second information comprises a type of the image, the type of the image comprising one of:
an x-ray radiographic image,
an ultrasound sonographic image,
a magnetic-resonance imaging image,
an endoscopic photograph,
an epidermal photograph, and
a nuclear emission radiographic image.

44. The system of claim 35, wherein the second information comprises a category of the image, the category comprising any one of or any combination of:
a two-dimensional image,
a three-dimensional image,
a surface image,
a cross-sectional image, and
a tomographic image in a set of tomographic images.

45. The system of claim 44, wherein the second information is obtained from the image or from additional information obtained by the computer processor separately from the medial records.

46. The system of claim 40,
wherein the processing using the first information determines an image-quality score for the first factor, and compares the image-quality score to a threshold value above which a reliable analysis cannot be made from the image, and
wherein the method further comprises generating and transmitting to the requester a rejection notification indicating that the image corresponding to the image-quality score was rejected from analysis due to low image quality, when the image-quality score is above the threshold value.

47. The system of claim 46, wherein the processing using the second information commences after the image-quality score is determined to be at or below the threshold value.

48. The system of claim 40, wherein the processing using the second information comprises determining the image to be any one or any combination of:
an external body region,
an internal body region,
an external body part,
an internal body part,
an internal organ,
an implanted object,
a prosthetic device, and
a skeletal part.

49. The system of claim 40, wherein the processing using the second information comprises determining whether the image includes an anomaly.

50. The system of claim 49, wherein the anomaly comprises any one of or any combination of:
a bone fracture,
a joint dislocation,
an abnormal surface contour of an internal organ,
an abnormal surface texture of an external body part,
an abnormal surface texture of an internal organ,
an abnormal inclusion of an opaque region in an internal organ,
an abnormal inclusion of an opaque internal region external to an internal organ,
a region of abnormal pigmentation on an external body part,
a region of abnormal surface contour of an external body part,
an abnormal shape of an internal organ,
an absence of an internal organ,
an abnormal size of an internal organ relative to another internal organ,
a dislocation of an implanted object, and
an absence of a skeletal part.

51. A non-transitory computer-readable storage medium storing a program that, when executed by a computer, causes the computer to perform a method for expediting screening of medical data, the method comprising:
obtaining a plurality of medical records, each of the medical records comprising a digitized image of a body region of a patient;
for each of the images:
performing a character-recognition process to locate at least one character in the image, the at least one character being one of or a combination of: a symbol character and a text character,
masking each of the located at least one character, to obtain a masked image,
performing an identification process on the masked image to identify the body region of the image,
performing an analysis routine on the masked image to determine a screening score, the analysis routine corresponding to the identified body region, and,
if the screening score determined by the analysis routine is within a normal range for the analysis routine, generating, using the computer processor, a normal notification indicating that the image is a normal image within a healthy range for the identified body region; and
automatically transmitting the normal notification to a requester.

52. The storage medium of claim 51, wherein the obtaining of the medical records comprises receiving the medical records transmitted by the requester.

53. The storage medium of claim 51, wherein the obtaining of the medical records comprises retrieving the medical records from a memory in which the medical records are deposited by the requester.

54. The storage medium of claim 51, wherein the analysis routine determines the screening score by combining a plurality of sub-scores resulting from a plurality of sub-routines of the analysis routine.

55. The storage medium of claim 51, wherein the normal notification is transmitted to the requester together with other normal notifications generated from the received medical records.

56. The storage medium of claim 51, wherein the performing of the character-recognition process to locate the at least one character in the image recognizes the at least one character in the image.

57. The storage medium of claim 56, wherein the character-recognition process recognizes the at least one character in the image to be alphanumeric text corresponding to the image.

58. The storage medium of claim 57, wherein the alphanumeric text is a word or a string of words corresponding to the image.

59. The storage medium of claim 56, wherein the analysis routine comprises:
identifying indicator information,
obtaining first and second information from the indicator information,
processing the image using the first information to determine a first factor,
processing the image using the second information to determine a second factor, and
calculating the screening score using at least the first and second factors.

60. The storage medium of claim 59,
wherein the first information of the indicator information comprises image-quality information, and
wherein the second information of the indicator information comprises the body region identified in the identification process.

61. The storage medium of claim 60, wherein the at least one character recognized in the character-recognition process is utilized in the identification process to identify the body region of the image.

62. The storage medium of claim 60, wherein the at least one character recognized in the character-recognition process is not utilized in the identification process to identify the body region of the image.

63. The storage medium of claim 60, wherein the identification process performs an object-contour routine on the image to identify contours of at least one object in the image.

64. The storage medium of claim 63, wherein, to identify the body region, the identification process performs a comparison routine to compare the contours or a portion of the contours of the at least one object in the image with one or more reference images stored in a memory accessible by the computer processor.

65. The storage medium of claim 59,
wherein the first factor is determined by comparing the first information to first reference information stored in a database accessible by the computer processor, and
wherein the second factor is determined by comparing the second information to second reference information stored in a database accessible by the computer processor.

66. The storage medium of claim 60, wherein the method further comprises:
evaluating the image to determine the image-quality information, the image-quality information comprising a quality value for any one of or any combination of:
a level of blurriness,
evidence of patient movement,
an appropriateness of magnification,
a correctness of imaging view,
a presence of a non-patient artifact,
an image-digitization artifact,
an overexposure condition, and
an underexposure condition.

67. The storage medium of claim 51,
wherein each of the medical records further comprises additional information, the additional information comprising any one of or any combination of:
a related previous medical image of the patient,
a medical history of the patient,
demographic information of the patient,
a previous diagnosis of the patient,
a physician comment regarding the patient, and
an upcoming medical test of the patient, and
wherein the analysis routine takes into account the additional information to determine the screening score.

68. The storage medium of claim 60, wherein the second information comprises a type of the image, the type of the image comprising one of:
an x-ray radiographic image,
an ultrasound sonographic image,
a magnetic-resonance imaging image,
an endoscopic photograph,
an epidermal photograph, and
a nuclear emission radiographic image.

69. The storage medium of claim 60, wherein the second information comprises a category of the image, the category comprising any one of or any combination of:
a two-dimensional image,
a three-dimensional image,
a surface image,
a cross-sectional image, and
a tomographic image in a set of tomographic images.

70. The storage medium of claim 69, wherein the second information is obtained from the image of from additional information obtained by the computer processor separately from the medial records.

71. The storage medium of claim 65,
wherein the processing using the first information determines an image-quality score for the first factor, and compares the image-quality score to a threshold value above which a reliable analysis cannot be made from the image, and
wherein the method further comprises generating and transmitting to the requester a rejection notification indicating that the image corresponding to the image-quality score was rejected from analysis due to low image quality, when the image-quality score is above the threshold value.

72. The storage medium of claim 71, wherein the processing using the second information commences after the image-quality score is determined to be at or below the threshold value.

73. The storage medium of claim 65, wherein the processing using the second information comprises determining the image to be any one or any combination of:
an external body region,
an internal body region,
an external body part,
an internal body part,
an internal organ,
an implanted object,
a prosthetic device, and
a skeletal part.

74. The storage medium of claim 65, wherein the processing using the second information comprises determining whether the image includes an anomaly.

75. The storage medium of claim 74, wherein the anomaly comprises any one of or any combination of:
a bone fracture,
a joint dislocation,
an abnormal surface contour of an internal organ,
an abnormal surface texture of an external body part,
an abnormal surface texture of an internal organ,
an abnormal inclusion of an opaque region in an internal organ,
an abnormal inclusion of an opaque internal region external to an internal organ,
a region of abnormal pigmentation on an external body part,
a region of abnormal surface contour of an external body part,
an abnormal shape of an organ,
an absence of an internal organ,
an abnormal size of an internal organ relative to another internal organ,
a dislocation of an implanted object, and
an absence of a skeletal part.

76. A computer-implemented method for screening medical data, comprising:
electronically obtaining a plurality of medical records provided by a requester, each of the medical records comprising an image of a body region of a patient; and,
for each of the images, utilizing a computer processor to:
perform automatically an identification process on the image to identify the body region,
select automatically an analysis routine to analyze the identified body region,
analyze automatically the medical image using the selected analysis routine to calculate a screening score corresponding to the image, and,
if the screening score corresponding to the image is equal to or below a predetermined threshold for the selected analysis routine, generate automatically a normal notification indicating that the image is a normal image within a healthy range for the identified body region.

77. The method of claim 76, wherein the obtaining of the medical records comprises receiving the medical records transmitted by the requester.

78. The method of claim 76, wherein the obtaining of the medical records comprises retrieving the medical records from a memory in which the medical records are deposited by the requester.

79. The method of claim 76, wherein the analysis routine determines the screening score by combining a plurality of sub-scores resulting from a plurality of sub-routines of the analysis routine.

80. The method of claim 76, the method further comprising:
automatically transmitting the normal notification to the requester.

81. The method of claim 80, wherein the normal notification is transmitted to the requester together with other normal notifications generated from the received medical records.

82. The method of claim 76, wherein the utilizing of the computer processor further comprises, if the screening score corresponding to the image is above the predetermined threshold value for the selected analysis routine, flagging the image for further analysis.

83. The method of claim 76, wherein the utilizing of the computer processor further comprises removing automatically, from further analysis, each image resulting in a normal notification.

84. The method of claim 76, wherein the utilizing of the computer processor further comprises obtaining automatically, from a memory accessible by the computer processor, the selected analysis routine.

85. The method of claim 76, wherein the identification process comprises:
identifying at least one character on the image, and
masking the at least one character from undergoing analysis by the selected analysis routine.

86. The method of claim 85, wherein the identification process further comprises:
performing a character recognition process on the at least one character to determine at least one recognized character.

87. The method of claim 86, wherein the identification process utilizes the at least one recognized character to identify the body region.

88. The method of claim 86, wherein the at least one character recognized in the character-recognition process is not utilized in the identification process to identify the body region.

89. The method of claim 86, wherein the identification process utilizes the at least one recognized character to identify a type of the image.

90. The method claim 89, wherein the type of the image comprises one of:
an x-ray radiographic image,
an ultrasound sonographic image,
a magnetic-resonance imaging image,
an endoscopic photograph,
an epidermal photograph, and
a nuclear-emission radiographic image.

91. The method of claim 85, wherein the identification process utilizes the at least one recognized character to identify the image as any one of or any combination of:
a two-dimensional image,
a three-dimensional image,
a surface image,
a cross-sectional image, and
a tomographic image in a set of tomographic images.

92. The method of claim 76, wherein the identification process performs an object-contour routine on the image to identify contours of at least one object in the image.

93. The method of claim 92, wherein, to identify the body region, the identification process performs a comparison routine to compare the contours of the at least one object in the image with one or more reference images stored in a memory accessible by the computer processor.

94. The method of claim 93, wherein a comparison result of the comparison routine identifies any one of or any combination of:
an external body region,
an internal body region,
an external body part,
an internal body part,
an internal organ,
an implanted object,
a prosthetic device, and
a skeletal part.

95. The method of claim 76,
wherein each of the medical records further comprises additional information, the additional information comprising any one of or any combination of:
a previous medical image of the patient,
a medical history of the patient,
demographic information of the patient,
a previous diagnosis of the patient,
a physician comment regarding the patient, and
an upcoming medical test of the patient, and
wherein the selected analysis routine utilizes data from the additional information to calculate the screening score corresponding to the image.

96. The method of claim 76, wherein the utilizing of the computer processor further comprises:
prior to the identification process, evaluating the image to determine a quality score for the image, the quality score taking into account any one of or any combination of:
a level of blurriness,
evidence of patient movement,
an appropriateness of magnification,
a correctness of imaging view,
a presence of a non-patient artifact,
an image-digitization artifact,
an overexposure condition, and
an underexposure condition,
if the quality score for the image is at or above a predetermined threshold value, generating a rejection notification indicating that the image was rejected for being of insufficient quality to enable a reliable analysis, and,
if the quality score for the image is below the predetermined threshold value, proceeding to perform the identification process.

97. A system for screening medical data, the system comprising:
a receiver connected to a communication network, the receiver structured to receive a plurality of medical records, each of the medical records comprising an image of a body region of a patient;
a computer processor coupled to a memory, the computer processor programmed to, for each of the images:
perform an identification process on the image to identify the body region,
select an analysis routine to analyze the identified body region,
analyze the medical image using the selected analysis routine to calculate a screening score corresponding to the image, and,
if the screening score corresponding to the image is equal to or below a predetermined threshold for the selected analysis routine, generate a normal notification indicating that the image is a normal image within a healthy range for the identified body region;
a transmitter connected to the communication network, the transmitter structured to transmit the normal notification to a requester.

98. The system of claim 97, wherein the receiver receives the medical records transmitted by the requester.

99. The system of claim 97, wherein the receiver retrieves the medical records from a memory in which the medical records are deposited by the requester.

100. The system of claim 97, wherein the analysis routine determines the screening score by combining a plurality of sub-scores resulting from a plurality of sub-routines of the analysis routine.

101. The system of claim 97, wherein the transmitter transmits the normal notification to the requester automatically.

102. The system of claim 101, wherein the normal notification is transmitted to the requester together with other normal notifications generated from the medical records.

103. The system of claim 97, wherein the computer processor is further utilized to, if the screening score corresponding to the image is above the predetermined threshold value for the selected analysis routine, flag the image for further analysis.

104. The system of claim 97, wherein the computer processor is further utilized to remove automatically, from further analysis, each image resulting in a normal notification.

105. The system of claim 97, wherein the computer processor is further utilized to obtain automatically, from a memory accessible by the computer processor, the selected analysis routine.

106. The system of claim 97, wherein the identification process:
identifies at least one character on the image, and
masks the at least one character from undergoing analysis by the selected analysis routine.

107. The system of claim 106, wherein the identification process:
performs a character recognition process on the at least one character to determine at least one recognized character.

108. The system of claim 107, wherein the identification process utilizes the at least one recognized character to identify the body region.

109. The system of claim 107, wherein the at least one character recognized in the character-recognition process is not utilized in the identification process to identify the body region.

110. The system of claim 107, wherein the identification process utilizes the at least one recognized character to identify a type of the image.

111. The system claim 110, wherein the type of the image comprises one of:
an x-ray radiographic image,
an ultrasound sonographic image,
a magnetic-resonance imaging image,
an endoscopic photograph,
an epidermal photograph, and
a nuclear-emission radiographic image.

112. The system of claim 106, wherein the identification process utilizes the at least one recognized character to identify the image as any one of or any combination of:
a two-dimensional image,
a three-dimensional image,
a surface image,
a cross-sectional image, and
a tomographic image in a set of tomographic images.

113. The system of claim 97, wherein the identification process performs an object-contour routine on the image to identify contours of at least one object in the image.

114. The system of claim 113, wherein, to identify the body region, the identification process performs a comparison routine to compare the contours of the at least one object in the image with one or more reference images stored in a memory accessible by the computer processor.

115. The system of claim 114, wherein a comparison result of the comparison routine identifies any one of or any combination of:
an external body region,
an internal body region,
an external body part,
an internal body part,
an internal organ,
an implanted object,
a prosthetic device, and
a skeletal part.

116. The system of claim 97,
wherein each of the medical records further comprises additional information, the additional information comprising any one of or any combination of:
a previous medical image of the patient,
a medical history of the patient,
demographic information of the patient,
a previous diagnosis of the patient,
a physician comment regarding the patient, and
an upcoming medical test of the patient, and
wherein the selected analysis routine utilizes data from the additional information to calculate the screening score corresponding to the image.

117. The system of claim 97, wherein the computer processor is further utilized to:
prior to the identification process, evaluate the image to determine a quality score for the image, the quality score taking into account any one of or any combination of:
a level of blurriness,
evidence of patient movement,
an appropriateness of magnification,
a correctness of imaging view,
a presence of a non-patient artifact,
an image-digitization artifact,
an overexposure condition, and
an underexposure condition,
if the quality score for the image is at or above a predetermined threshold value, generate a rejection notification indicating that the image was rejected for being of insufficient quality to enable a reliable analysis, and,
if the quality score for the image is below the predetermined threshold value, proceed to perform the identification process.

118. A non-transitory computer-readable storage medium storing a program that, when executed by a computer, causes the computer to perform a method for screening medical data, the method comprising:
electronically receiving a plurality of medical records, each of the medical records comprising an image of a body region of a patient;
for each of the images:
perform an identification process on the image to identify the body region,
select an analysis routine to analyze the identified body region,
analyze the medical image using the selected analysis routine to calculate a screening score corresponding to the image, and,
if the screening score corresponding to the image is equal to or below a predetermined threshold for the selected analysis routine, generate a normal notification indicating that the image is a normal image within a healthy range for the identified body region; and
automatically transmitting the normal notification to a requester.

119. The storage medium of claim 118, wherein the obtaining of the medical records comprises receiving the medical records transmitted by the requester.

120. The storage medium of claim 118, wherein the obtaining of the medical records comprises retrieving the medical records from a memory in which the medical records are deposited by the requester.

121. The storage medium of claim 118, wherein the analysis routine determines the screening score by combining a plurality of sub-scores resulting from a plurality of sub-routines of the analysis routine.

122. The storage medium of claim 118, wherein the method further comprises:
automatically transmitting the normal notification to the requester.

123. The storage medium of claim 122, wherein the normal notification is transmitted to the requester together with other normal notifications generated from the received medical records.

124. The storage medium of claim 118, wherein the utilizing of the computer processor further comprises, if the screening score corresponding to the image is above the predetermined threshold value for the selected analysis routine, flagging the image for further analysis.

125. The storage medium of claim 118, wherein the utilizing of the computer processor further comprises removing automatically, from further analysis, each image resulting in a normal notification.

126. The storage medium of claim 118, wherein the utilizing of the computer processor further comprises obtaining automatically, from a memory accessible by the computer processor, the selected analysis routine.

127. The storage medium of claim 118, wherein the identification process comprises:
identifying at least one character on the image, and
masking the at least one character from undergoing analysis by the selected analysis routine.

128. The storage medium of claim 127, wherein the identification process further comprises:
performing a character recognition process on the at least one character to determine at least one recognized character.

129. The storage medium of claim 128, wherein the identification process utilizes the at least one recognized character to identify the body region.

130. The storage medium of claim 128, wherein the at least one character recognized in the character-recognition process is not utilized in the identification process to identify the body region.

131. The storage medium of claim 128, wherein the identification process utilizes the at least one recognized character to identify a type of the image.

132. The storage medium claim 131, wherein the type of the image comprises one of:
an x-ray radiographic image,
an ultrasound sonographic image,
a magnetic-resonance imaging image,
an endoscopic photograph,
an epidermal photograph, and
a nuclear-emission radiographic image.

133. The storage medium of claim 127, wherein the identification process utilizes the at least one recognized character to identify the image as any one of or any combination of:
a two-dimensional image,
a three-dimensional image,
a surface image,
a cross-sectional image, and
a tomographic image in a set of tomographic images.

134. The storage medium of claim 118, wherein the identification process performs an object-contour routine on the image to identify contours of at least one object in the image.

135. The storage medium of claim 134, wherein, to identify the body region, the identification process performs a comparison routine to compare the contours of the at least one object in the image with one or more reference images stored in a memory accessible by the computer processor.

136. The storage medium of claim 135, wherein a comparison result of the comparison routine identifies any one of or any combination of:
an external body region,
an internal body region,
an external body part,
an internal body part,
an internal organ,
an implanted object,
a prosthetic device, and
a skeletal part.

137. The storage medium of claim 118,
wherein each of the medical records further comprises additional information, the additional information comprising any one of or any combination of:
a previous medical image of the patient,
a medical history of the patient,
demographic information of the patient,
a previous diagnosis of the patient,
a physician comment regarding the patient, and
an upcoming medical test of the patient, and
wherein the selected analysis routine utilizes data from the additional information to calculate the screening score corresponding to the image.

138. The storage medium of claim 118, wherein the utilizing of the computer processor further comprises:
prior to the identification process, evaluating the image to determine a quality score for the image, the quality score taking into account any one of or any combination of:
a level of blurriness,
evidence of patient movement,
an appropriateness of magnification,
a correctness of imaging view,
a presence of a non-patient artifact,
an image-digitization artifact,
an overexposure condition, and
an underexposure condition,
if the quality score for the image is at or above a predetermined threshold value, generating a rejection notification indicating that the image was rejected for being of insufficient quality to enable a reliable analysis, and,
if the quality score for the image is below the predetermined threshold value, proceeding to perform the identification process.
